(19) United States
(12) Patent Application Publication
(10) Pub. No.: US 2024/0254426 A1
(43) Pub. Date: Aug. 1, 2024

(54) SYSTEMS, DEVICES, AND METHODS FOR SCHEDULING WORKFLOWS FOR AUTOMATED CELL THERAPY MANUFACTURING

(71) Applicant: Cellares Corporation, South San Francisco, CA (US)

(72) Inventors: Marc ELPEL, Poulsbo, WA (US); Stephen KULL, South San Francisco, CA (US); Daniele MALLEO, Menlo Park, CA (US); Howard SEAY, South San Francisco, CA (US)

(21) Appl. No.: 18/412,350

(22) Filed: Jan. 12, 2024

Related U.S. Application Data
(60) Provisional application No. 63/438,925, filed on Jan. 13, 2023.

(51) Int. Cl. C12M 1/36; G01N 35/00

(52) U.S. Cl. CPC C12M 41/48; G01N 35/0092

(57) ABSTRACT

Described herein are systems, devices, and methods for high-throughput manufacturing of cell products for biomedical applications using automated systems executing parallel workflows. A method for scheduling a cell processing cartridge for loading into a cell processing system may include determining a system configuration, determining a configuration for each of a plurality of cell processing cartridges of the system, and providing a loading schedule for loading a first cartridge of the plurality of cartridges into the system. A method for monitoring contention of a cell processing system processing a plurality of cell samples in parallel may include loading the plurality of cell samples into the system and determining contention for each of a plurality of processing instruments before and after a processing operation is executed by the system. The contention may be based on a number of cell samples that need the instrument during a time period.

### CROSS-REFERENCE TO RELATED APPLICATIONS

This application claims the benefit of U.S. Provisional Patent Application No. 63/438,925, filed on Jan. 13, 2023, which is incorporated by reference herein in its entirety for all purposes.

### TECHNICAL FIELD

Devices, systems, and methods herein relate to high-throughput manufacturing of cell products for biomedical applications using automated systems executing parallel workflows.

### BACKGROUND

Cell processing generally involves collecting and manufacturing cells for therapeutic use. For example, a cell product may be used to achieve a clinical response in a patient. Existing processing systems may define a multi-step workflow that combines automated cell processing systems devices with cumbersome manual operations performed in expensive biosafety cabinets and/or cleanrooms. Additionally, conventional systems may operate with only a single cell processing unit (e.g., a system configured to interface with a single cartridge carrying a single cell sample), resulting in low throughput. In contrast, automated (e.g., fully automated) multi-cartridge cell processing systems may require little to no operator interaction and may provide additional benefits such as decreased overhead, high-throughput manufacturing, end-to-end process flexibility, process robustness, and process scalability.

However, multi-cartridge systems may also experience contention within the automated cell therapy system due to overlapping requirements for system resources, leading to workflow delays or restarts. Excess delays due to contention during critical portions of a workflow may lead to unacceptable process outcomes that risk sample viability, and therefore risk patient treatment (e.g., treatment timing, treatment cost, etc.).

To optimize the high-throughput potential of multi-cartridge systems, managing and reducing contention within these systems is crucial. For example, a tool for scheduling consumable cartridges into an automated cell manufacturing system and continuously monitoring system and resource requirements for each cartridge being processed may ensure that the patient samples will not be negatively impacted by contention issues due to parallel processing. Accordingly, novel methods for managing and reducing contention within multi-cartridge automated cell processing systems may be desirable.

### SUMMARY

Described herein are systems, devices, and methods useful for cell processing, including high-throughput manufacturing of cell products for biomedical applications using automated systems executing parallel workflows.

A method for cell processing may first include determining a system configuration of a cell processing system configured to process a plurality of cartridges in parallel. Each of the plurality of cartridges may be configured to carry a cell sample and to be loaded into a workcell of the cell processing system. Next, the method may include determining a cartridge configuration for each of the plurality of cartridges and providing a loading schedule for loading a first cartridge of the plurality of cartridges into the workcell. The loading schedule may be based on the system configuration and the cartridge configuration for each of the plurality of cartridges. The loading schedule may indicate one or more time periods that are not available for loading the first cartridge into the workcell. Additionally, the method may further include loading the first cartridge into the workcell based on the loading schedule. In some variations, the loading schedule may be provided via a user interface of the cell processing system. Determining the cartridge configuration may include padding data of the cartridge configuration to compensate for one or both of workflow process variability and cartridge loading variability. Further, the method may include determining an amount of contention within the cell processing system, and the loading schedule may be further based on the amount of contention determined.

The system configuration may define a quantity of one or more subsystems of the cell processing system, which may be housed within the workcell of the cell processing system. In some variations, the one or more subsystems may include one or more of a materials handling system, a sterile liquid transfer system, a sterility system, a bioprocessing system, and a quality control system. In some variations, the system configuration may further define an operational time constraint for at least one of the one or more of subsystems of the cell processing system.

The cartridge configuration may include one or both of a loading configuration and a workflow configuration. The loading configuration may include a length of time for loading a cartridge into the workcell. Moreover, the workflow configuration may include a simulated cell processing workflow for a cartridge, and wherein the simulated cell processing workflow is based on a process design plan for the cartridge and one or more sample intake parameters for the cartridge. The one or more sample intake parameters may include one or more of a sample cell type, a sample collection time, a transport time for the sample, a processing facility arrival time for the sample, a sample temperature, patient age, patient sex, donor age, and donor sex.

Additionally, in some variations, the plurality of cell processing cartridges may include at least one second cartridge, where the first cartridge may not be scheduled for processing within the cell processing system, and where the at least one second cartridge may be scheduled for processing within the cell processing system. The method may further include scheduling the first cartridge for processing within the cell processing system based on the loading schedule.

Another method for cell processing may first include loading a plurality of cell samples into a cell processing system for parallel processing, where the cell processing system may include a plurality of processing instruments. Next, the method may include determining an amount of contention for each of the plurality of instruments before and after each of a plurality of cell processing operations are executed by the cell processing system. The amount of contention may be based on a number of cell samples that need the instrument during a time period. Finally, the method may include generating a notification, via a user interface of the cell processing system, indicative of excess contention when the number of cell samples that need the instrument during the time period is greater than two. In some variations, the method may further include identifying a prioritized call sample to use the instrument based on a weight of instrument contention determined for each cell sample that needs the instrument during the time period, and the prioritized cell sample may have a largest weight of instrument contention. For each cell sample, the weight of instrument contention may be based on a current cell processing operation dwell time and a subsequent cell processing operation execution time.

### BRIEF DESCRIPTION OF THE DRAWINGS

FIG. 1A is a block diagram of an illustrative variation of a cell processing system. FIG. 1B is a block diagram of an illustrative variation of a cartridge that may be used with the cell processing system of FIG. 1A.

FIG. 1C depicts a perspective view of an illustrative variation of a cell processing system.

FIG. 1D depicts a sectional view of an illustrative variation of a workcell that may be used with the cell processing system herein.

FIG. 2 is a flow chart illustrating two exemplary procedures for resolving contention of a cell processing system.

FIG. 3 is a flow chart illustrating an exemplary variation of a method for cell processing.

FIG. 4 is a flow chart illustrating an exemplary variation of another method for cell processing.

FIG. 5 is an exemplary variation of a cell processing workflow simulated by a cell processing system.

FIGS. 6A-6B are exemplary variations of a loading schedule for a cell process cartridge provided by a cell processing system.

FIGS. 7A-7B are exemplary variations of a loading schedule for a cell process cartridge provided by a cell processing system.

### DETAILED DESCRIPTION

### Definitions

As used herein, sterile should be understood as a non-limiting description of some variations, an optional feature providing advantages in operation of certain systems and methods of the disclosure. Maintaining sterility is typically desirable for cell processing but may be achieved in various ways, including but not limited to providing sterile reagents, media, cells, and other solutions; sterilizing cartridge(s) and/or cartridge component(s) after loading (preserving the cell product from destruction); and/or operating the system in a sterile enclosure, environment, building, room, or the like. Such user or system performed sterilization steps may make the cartridge or cartridge components sterile and/or preserve the sterility of the cartridge or cartridge components.

As used herein, the singular forms “a”, “an” and “the” include plural referents unless the context clearly dictates otherwise. “And” as used herein is interchangeably used with “or” unless expressly stated otherwise.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0)%). When used in conjunction with a range or series of values, the term “about” applies to the endpoints of the range or each of the values enumerated in the series, unless otherwise indicated. As used in this application, the terms “about” and “approximately” are used as equivalents.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” “below,” and words of similar import, when used in this application, shall refer to this application as a whole and not to any particular portions of the application.

Parallel processing of cartridges containing a cell sample within a cell processing system and may cause internal contention within the system due to resource competition (e.g., for reagents, components and/or functions of the cell processing system). This contention may cause one or more of the cartridges to delay or even restart a cell processing workflow, which may risk sample viability, and therefore risk patient treatment (e.g., treatment timing, treatment cost, etc.). Accordingly, as will be described herein, scheduling cartridges for parallel processing and resolving system contention due to parallel processing in real-time may allow multi-cartridge processing systems to maintain high throughout without risking sample quality.

Scheduling multiple cartridges for processing may present challenges. For example, in some variations, scheduling may be based on allowing random-access cartridge loading into a system cartridge. However, allowing for random loading of cartridges may result in random high-contention situations downstream in processing, which may risk patient sample viability. As another example, cartridge processing scheduling based only on contention prevention with no compensation for process deviation and/or load variability may result in higher overall process variability, and therefore longer periods of time during which cartridges may not be loaded into a cell processing system. Thus, scheduling the processing of multiple cartridges within an automated cell processing system may be difficult, and may necessitate a combination of inputs, calculations of contention, and feedback control to ensure that cartridges may be added to the system without inducing significant contention.

Accordingly, the present disclosure provides a “scheduler” application or tool which may be configured to schedule loading (e.g., asynchronous loading) of cartridges into an automated cell processing system utilizing a combination of inputs, contention calculations, and feedback control and may be used with an automated cell processing system to minimize risk of cell samples being negatively impacted by parallel processing contention issues. Additional variations, features, and advantages of the invention will be apparent from the following detailed description and through practice of the invention.

### 1. Cell Processing System

The cell processing systems herein may be fully integrated and may be configured to autonomously perform every step of a cell processing workflow.

The cell processing systems described herein may be configured to perform one or more cell processing steps in a workcell. The workcell may include a closed, automated environment, which may be configured to maintain a sterile environment. The workcell may receive a cartridge therein and perform one or more cell processing steps on cells in a cell solution (e.g., cell suspension) contained within the cartridge. For example, the cell processing system may include a workcell having a plurality of subsystems (each made up of one or more instruments or components) that may each be configured to independently perform one or more cell processing steps to the cells and/or cell solution, and a robot capable of moving the cartridge within the workcell (e.g., between one or more bays configured to receive a cartridge). The robot and/or other instruments may be configured to automatically operate such that operator assistance may not be required at any point during the workflow. For example, the robot may receive the cartridge and move the cartridge between locations (e.g., instruments, bays, storage vaults, feedthroughs) within the workcell according to a pre-programmed workflow, where each location may be associated with one or more cell processing steps. After performing one or more cell processing steps of the pre-programmed workflow, the workcell may be configured to transfer the cartridge out of the workcell (e.g., via the robot). Additionally, or alternatively, at least a portion of the cell solution may be transferred (e.g., via a fluid device or a fluidic manifold) to a second cartridge.

The cell solution (e.g., cell suspension, cell sample) described herein may contain cells that may be processed for subsequent use in cell therapies. The cell solution may include cells (e.g., allogeneic cells) in a fluid, such as a media (e.g., cell culture media). The cell solution may include cells from the same or different donors. Cells from the same donor may be split between one or more cartridges, such that separate cell processing steps may be performed on each of the cartridges and increase the overall throughput of the cell processing system described herein. The cell solution may be transferred to the cartridge prior to loading the cartridge into the workcell, such as by operating personnel. In some variations, the cartridge may be empty when loaded into the workcell such that the workcell may transfer a cell solution to the cartridge. In some variations, the cells from two or more cartridges may be combined according to a pre-determined ratio, which may correspond to an intended therapeutic treatment for a patient.

Further, the cell processing systems described herein may advantageously be configured to schedule parallel processing of a plurality of cell samples, as well as manage contention within the system due to parallel processing by monitoring the system contention due to numerous (e.g., more than 2, 3, 4, 5, 6, 7, 8, 9, 10, etc.) cartridges requiring use of at least a portion of a subsystem (e.g., an instrument of the subsystem), and resolving identified areas of high contention.

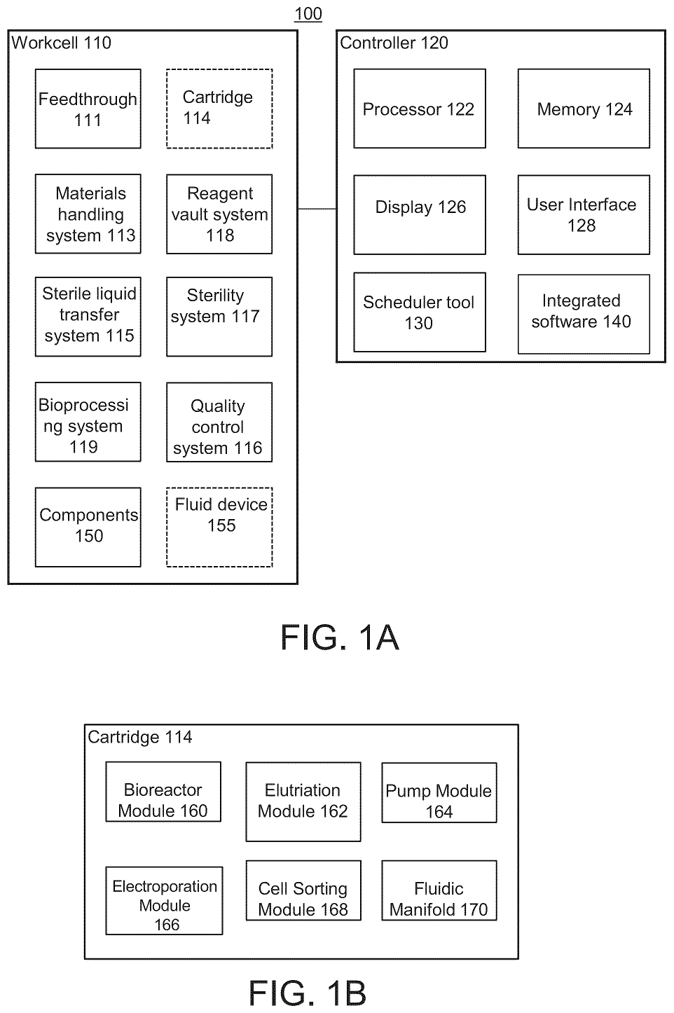

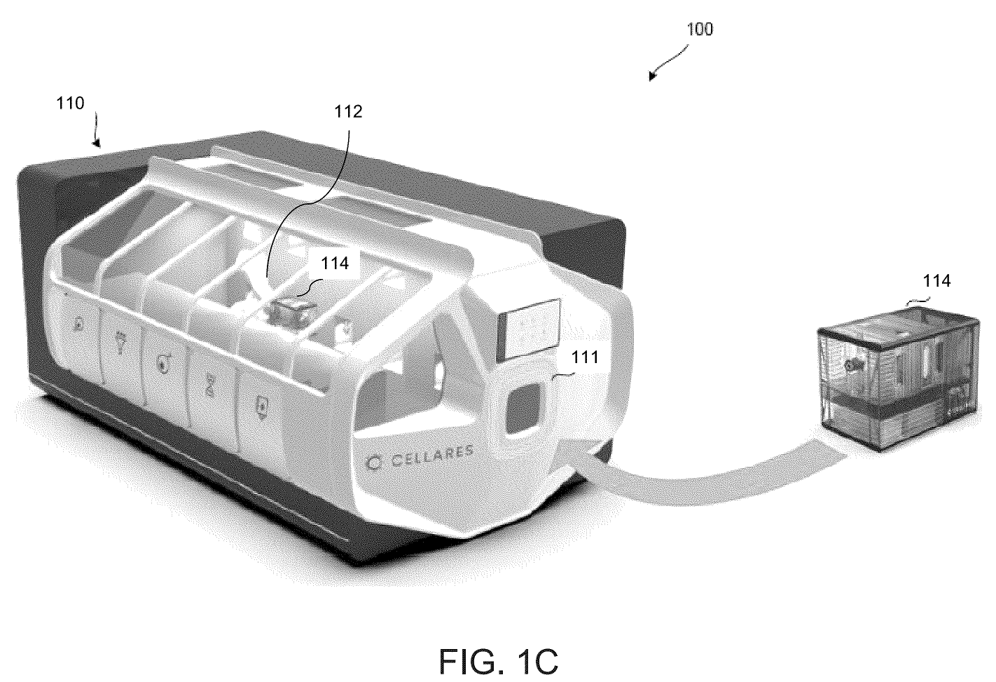

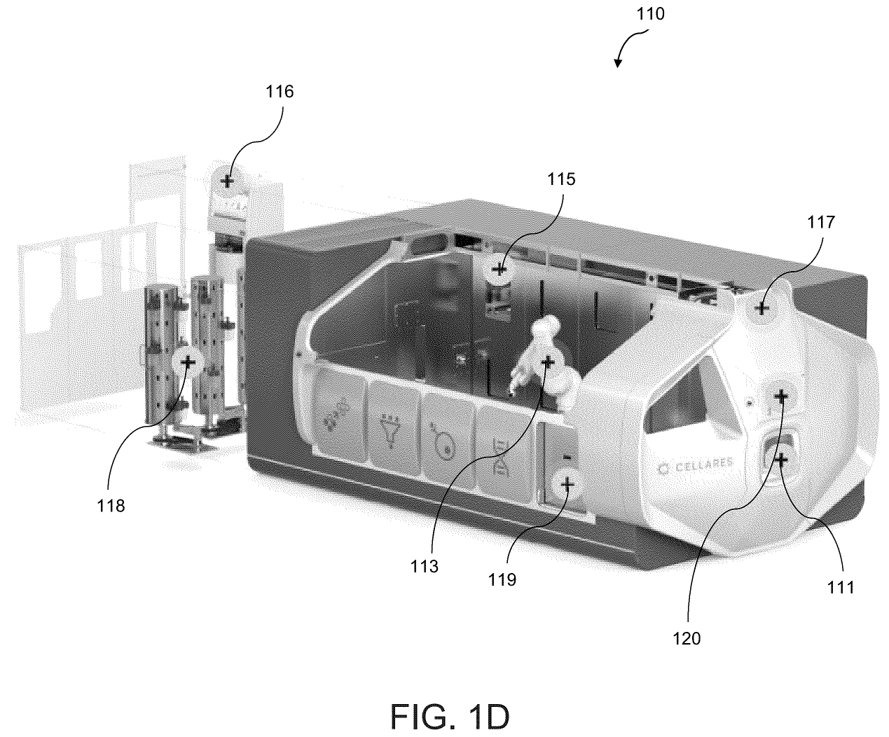

An exemplary cell processing system (CPS) for use with the automated devices, systems, and methods herein is shown in FIG. 1A. Shown there is a block diagram of a cell processing system (CPS) 100, which may generally include workcell 110, cartridge 114 (which may be loaded into the workcell 110), and controller 120. The workcell 110 may include one or more components and subsystems (having one or more components or instruments) including feedthrough 111, materials handling system (MHS) 113, reagent vault system (RVS) 118, sterile liquid transfer system (SLTS) 115, sterility system (SS) 117, bioprocessing system (BPS) 119, quality control system (QCS) 116, components 150, and fluid device 155. Cartridge 114 and fluid device 155, which may be provided outside of the workcell 110 and may be used within the workcell 110, are illustrated in dashed lines. The cartridge 114 may be reusable or disposable. Generally, the workcell 110 may be configured for long term use. One advantage of a split module/instrument design including a workcell and cartridge may be to retain expensive components within subsystems of the workcell 110 while less expensive components may reside in the cartridge 114. The components, such as components 150, may include one or more motors, sensors (e.g., cameras), heaters, lasers, pumps, and the like.

The cartridge 114 may include one or more modules defined by a group of associated components (mechanical and/or electrical) configured to perform a particular cell processing operation in conjunction with the workcell 110. Referring to FIG. 1B, as shown, the cartridge 114 may include one or more of bioreactor module 150, elutriation module 162, pump module 164, electroporation module 166, cell sorting module 168 (e.g., magnetic activated cell sorting (MACS)), and fluidic manifold 170.

FIG. 1C depicts a perspective view of an illustrative variation of a CPS 100 including workcell 110 and cartridge 114. The cartridge 114 may be loaded into the workcell 110 via a feedthrough 111. In some variations, the feedthrough 111 may be a first of a plurality of feedthroughs 111. Generally, cell processing may involve moving the cartridge 114, configured to carry a cell sample, between a plurality of components and subsystems of the workcell 110, as described herein. One or more of the workcell subsystems or modules (e.g., modules of the BPS 119, etc.) may be configured to interface with the cartridge 114 to perform processing steps on cells within the cartridge 114. In some variations, a plurality of cell processing steps may be performed within the cartridge 114. For example, the robot 112 (e.g., a robotic arm) shown in FIG. 1C, which may be part of the MHS 113 of FIG. 1A, may be configured to move the cartridge 114 between subsystems of the workcell 110. Each module may be configured to perform a particular cell processing step when coupled to (e.g., interfaced and/or engaged with) a corresponding instrument module within the cartridge 114. In some variations, each subsystem or module of the workcell 110 may include a receiving bay or dock for one of a plurality of cartridges 114 such that a plurality of cartridges 114 may be housed within the workcell 110 and plurality of subsystems of the workcell 110 may be in use at any given time. Further, each cartridge 114 may have a unique workflow or process design, and the workcell 110 may be configured to perform a plurality of different workflows in parallel.

In some variations, a plurality of cartridges 114 may be inserted into the workcell 110 and undergo one or more cell processing operations in parallel. For example, the workcell 110 may receive between 1 and 30 cartridges 114 for processing, such as between 1 and 16 cartridges (e.g., 1 cartridge, 2 cartridges, 3 cartridges, 4 cartridges, 5 cartridges, 6 cartridges, 7 cartridges, 8 cartridges, 9 cartridges, 10 cartridges, 11 cartridges, 12 cartridges, 13 cartridges, 14 cartridges, 15 cartridges, or 16 cartridges). As discussed herein, a performance of the workcell 110 may depend its ability to resolve internal contention due to multi-processing of cell samples. Thus, the workcell 110, during multi-product manufacturing, the workcell 110 may have an optimal performance at below 100% cartridge capacity, such between about 50% to 95% capacity, between about 60% to 90% capacity, between about 70% to 85% capacity, between about 75% to 80% capacity, about 80% capacity, or about 90% capacity. In other variations, the workcell 110 may perform optimally at about 100% cartridge capacity during multi-product manufacturing.

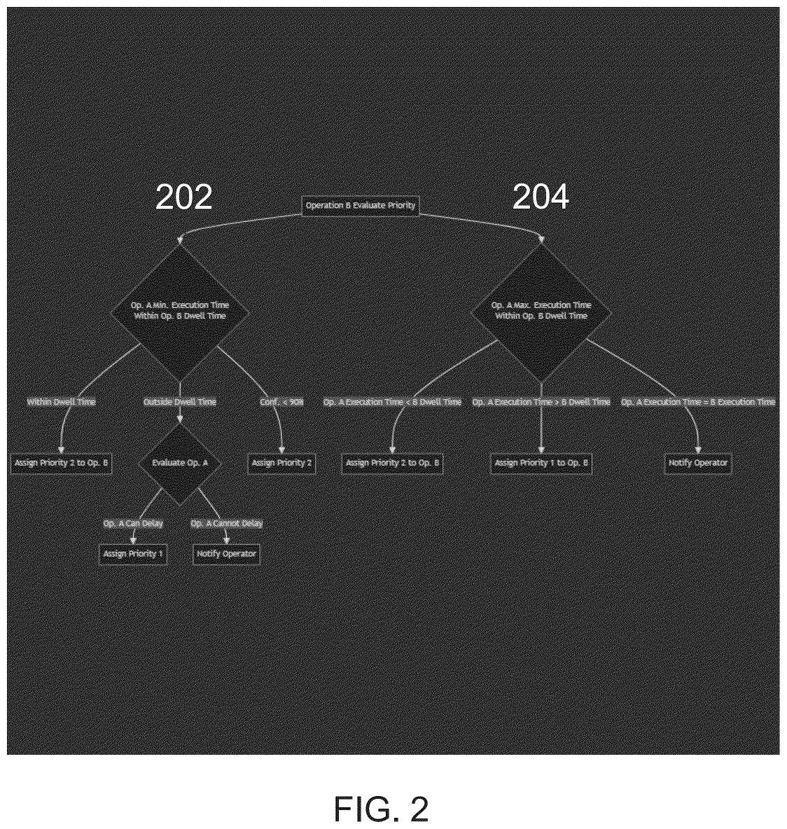

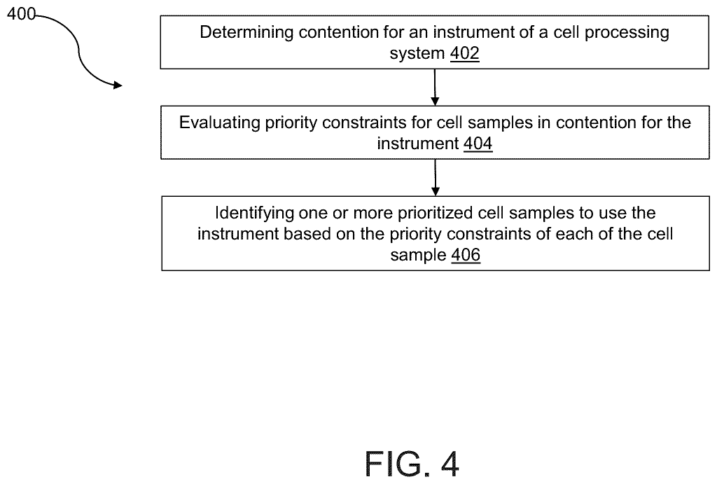

Referring again to FIG. 1A, the controller 120 of CPS 100 may include one or more of a processor 122, a memory 124, a display 126, a user interface 128, a scheduler tool 130, and integrated software 140 (e.g., MES, ERP, LIMS). In some variations, the controller 120 may be configured to communicate (e.g., via a wired or wireless connection) with one or more components of the CPS 100 (e.g., QCS 116), one or more remote devices (e.g., a remote control, such as a mobile device), and/or one or more additional CPSs 100 (e.g., a second controller 120 of a second CPS 100 and/or a third controller 120 of a third CPS 100, etc.). In one example, a first controller 120 of a first CPS 100 may be communicably coupled with a second controller 120 of a second, proximal CPS 100 via a shared network. As such, the two CPSs 100 may be configured to jointly schedule and/or manage contention within the greater system during parallel processing of a plurality of cartridges. In some variations, if, using the scheduler tool 130, the first CPS 100 identifies high contention within the workcell (e.g., two cartridges may require a same resource for a period of time) and cannot resolve the contention (e.g., neither of the two cartridges is determined to have a prioritized need for the resource relative to the other), then the second CPS 100 may be able to take in and perform a subsequent cell processing operation on one of the two competing cartridges. Such a scenario may arise if, for example, two cartridges 114 require a same resource of the workcell 110 for an (at least partially) overlapping period of time, and the CPS 100 is unable to determine the relative priority of the two cartridges 114 (e.g., each cartridge is determined to have an equal need for the resource). Monitoring and resolving contention during multi-product manufacturing will be described in detail herein with respect to FIG. 2 and FIG. 4.

Any suitable cell processing workflow may be performed using the systems and devices described herein, and may include steps such as growing, enriching, selecting, sorting, expanding, activating, transducing, electroporating, washing, and the like, in order to meet a desired specification and/or quality of a cell product (e.g., a desired cell count). In some variations, a method of processing a solution containing a cell product includes the steps of digesting tissue using an enzyme reagent to release a select cell population into solution, enriching cells using a CCE instrument, washing cells using the CCE instrument, selecting cells in the solution using a selection instrument, sorting cells in the solution using a sorting instrument, differentiating or expanding the cells in a bioreactor, activating cells using an activating reagent, electroporating cells, transducing cells using a vector, and finishing a cell product.

Generally, a cell processing workflow may take between about 9 days to about 2 weeks. The type, number, and length of cell processing operations (including repeated operations) in the workflow may vary greatly for different cell samples. The workflow for each cell sample may depend on initial parameters such as cell type (e.g., autogenic versus allogenic), sample collection time, sample transport time (e.g., to a facility running a CPS such as CPS 100), sample arrival time (e.g., at a facility running a CPS such as CPS 100), sample temperature (e.g., frozen vs. thawed), patient or donor age, and patient or donor sex. Estimated initial parameters such as expected recovery from cell separation and/or expected recovery time may also inform the process design for a cell sample. Further, as discussed herein, manufacturing process variability (e.g., due to operator variability and/or contention among cell samples within a manufacturing unit) may change the workflow for a given sample. Accordingly, the CPS 100 may be configured to perform a unique workflow for each cell sample during parallel processing of two or more cell samples (e.g., carried by two or more corresponding cartridges 114). That is, a workflow having a unique type and series of operations may be defined for each cell sample, and the CPS 100 may synchronously process a plurality of cell samples according to a plurality of corresponding unique workflows.

### 1.1 Workcell

The workcell 110 may include a fully, or at least partially, enclosed housing inside which one or more cell processing steps may be performed in a fully, or at least partially, automated process. FIG. 1D depicts a sectional view of an illustrative variation of a workcell 110 including the feedthrough 111, the materials handling system (MHS) 113, the reagent vault system (RVS) 118, the sterile liquid transfer system (SLTS) 115, the sterility system (SS) 117, the bioprocessing system (BPS) 119, the quality control system (QCS) 116, the components 150, and the fluid device 155.

The feedthrough 111 may be an entry and/or exit point for a cartridge (e.g., cartridge 114 of FIGS. 1A-1C) relative to the workcell 110. The feedthrough 111 may be configured to receive a plurality of cartridges asynchronously for multi-product concurrent manufacturing within the workcell 110. The feedthrough 111 may be operatively coupled (e.g., via a wired or wireless connection) to the SS to sterilize a cartridge as it is docked within the feedthrough 111. In some variations, the workcell 110 may include a plurality of feedthroughs, such as between 2 and 10 feedthroughs, between 3 and 8 feedthroughs, or between 4 and 6 feedthroughs. For example, the workcell 110 may include a first feedthrough 111.

The MHS 113 may be an automated transferring system having one or more mechanisms for transferring cartridges and fluid devices throughout the workcell 110 (e.g., between feedthroughs, the RVS, the BPS, the SLTS, etc.). In some variations, the MHS 113 may include a robotic arm (or a plurality of robotic arms) having at least one end for releasably coupling to a cartridge and/or a fluid device (e.g., fluid device 155) for transfer. Accordingly, the MHS 113 may be configured to interact with one or more of the feedthrough 111, the RVS 118, the SLTS 115, and/or the BPS 119. Additionally, the MHS 113 may be communicably coupled to one or more components of a cell processing system (e.g., CPS 100 of FIGS. 1A-1C), such as the controller 120.

The RVS 118 may include one or more reagent vaults configured to store reagents within fluid devices, such as fluid device 155. In some variations, the reagent vault may be refrigerated. Nonlimiting examples of reagents stored within a reagent vault of the RVS 118 may include culture media, buffer, cytokines, proteins, enzymes, polynucleotides, transfection reagents, non-viral vectors, viral vectors, antibiotics, nutrients, cryoprotectants, solvents, cellular materials, and pharmaceutically acceptable excipients. Additionally, or alternatively, waste may be stored in a reagent vault of the RVS 118, or in the fluid device 155, which may be within the a reagent vault.

The RVS 118 may communicably coupled to the SS to enable sterilization of one or more fluid devices therein. Additionally, the RVS 118 may be communicably coupled to one or more components of a cell processing system (e.g., CPS 100 of FIGS. 1A-IC), such as the controller (e.g., controller 120 via scheduler tool 130 of FIG. 1A) for reagent scheduling, inventory, and/or supply chain management. In some variations, the RVS 118 may include an access point through which a reagent may be delivered or replaced during cell processing. For example, an operator may deliver a reagent through the access point of the RVS 118 at any time during cell processing.

The fluid device 155 may be a sterile liquid transfer device (SLTD). However, it should be appreciated that the fluid device 155 may be configured to transfer any fluid (including liquids), whether sterile or not. In general, the fluid device 155 may interact with the SLTS 115, which may facilitate mating between a cartridge (e.g., cartridge 114 of FIGS. 1A-1C) and the fluid device 155. Additionally, the SLTS 115 may perform all sterile liquid transfer operations within the system, including reagent addition and sampling. As such, the SLTS 115 may be an in-demand (contentious) resource within the workcell 110.

The workcell 110 may include a one or more SLTSs 115, such as 1, 2, 3, 4, 5, 6, 7, 8, 9, 10, or more than 10 SLTSs 115. In some variations, the workcell 110 may include 4 SLTSs 115.

The SS 117 may be configured to sterilize cartridges and fluid devices 155 such that the workcell 110 may maintain an internal cleanroom environment. In some variations, the SS 117 may be configured to perform a sterilization procedure (e.g., vaporized hydrogen peroxide (VHP) processing, ionized hydrogen peroxide decontamination) as a cartridge enters or exits the workcell (e.g., via the feedthrough 111) and/or as the fluid device 155 enters or exits the RVS 118.

The BPS 119 may include one or more workcell modules configured to interface and/or engage with (electrically and/or mechanically) corresponding cartridge modules to carry out a cell processing operation. The one or more workcell modules may include an elutriation module, a cell sorting module (e.g., a magnetic-activated cell sorting module), an electroporation module, and a bioreactor. The workcell 110 may include one or more BPSs 119, such as between 1 and 30 BPSs, between 2 and 28 BPSs, between 4 and 26 BPSs, between 6 and 24 BPSs, between 8 and 22 BPSs, between 10 and 20 BPSs, between 12 and 18 BPSs, or between 14 and 16 BPSs (e.g., 16 BPSs). In some variations, the workcell 110 may perform optimal parallel processing (e.g., experience minimal contention) for a plurality of cell samples when fewer than all of the BPSs 119 (e.g., about 80% or about 90% of the number of BPSs 119) are engaged with a cartridge. For example, for a workcell having 16 BPSs, an optimal capacity of the workcell may be 14 cartridges (each for processing within one of 14 BPSs).

The QCS 116 may be configured to perform automated sampling and analysis of a cell sample (batch, cell product) during processing. In some variations, the QCS 116 may include an integrated cell culture analyzer configured to measure and/or estimate a cell count and/or cell viability of a sample. Additionally, or alternatively, sample containers may be collected from the workcell 110, and an operator or other system may perform the analysis. Moreover, the QCS 116 may be configured to generate (e.g., auto-generate) and update records of a cell sample as it progresses through manufacturing. The batch records may be generated and/or updated via real-time process monitoring by the QCS 116. That is, first, the QCS 116 may collect (e.g., via the SLTS 115) a sample, analyze the sample, and update records associated with the sample. Next, the QCS 115 may (1) display the records (e.g., digitally via a display) for an operator, and/or (2) transmit the records to a scheduler tool (e.g., scheduler tool 130 of FIG. 1A) so that the real-time processing results may inform scheduling and/or contention resolution within the system.

In some variations, one or more components and/or subsystems of the workcell 110 may include one or more fixed or operational time constraints defined as an amount of time required for the component or subsystem to complete a task. Such fixed time constraints may impact scheduling of multi-process cell manufacturing, as discussed herein. As an example, the MHS 113 may have one or more fixed time constraints for transferring (e.g., via a robotic arm) a cartridge and/or a fluid device, such as a minimum time required to transfer a cartridge 114 or a fluid device 155 from a first subsystem of the workcell (e.g., from the feedthrough 111 or RVS 118) to a second subsystem of the workcell (e.g., to the BPS 119).

### 1.2 Cartridge

The cell processing systems described herein may include one or more cartridges having one or more modules configured to interface with, or releasably couple to, one or more instruments within a workcell of a cell processing system. In general, each cartridge 114 within a cell processing system may be used to manufacture a unique cell therapy product.

Some or all of the modules of a cell processing cartridge may be integrated in a fixed configuration within the cartridge, though they need not be. For example, one or more of the modules may be configurable or moveable (e.g., by an operator, controller, and/or robot of the workcell) within the cartridge, permitting various formats of cartridges to be assembled. For example, the cartridge may be a single, closed unit with fixed components for each module, or the cartridge may contain configurable modules coupled by configurable fluidic, mechanical, optical, and electrical connections. In some variations, one or more sub-cartridges, each containing a set of modules, may be used to perform various cell processing workflows. The modules may each be provided in a distinct housing or may be integrated into a cartridge or sub-cartridge with other modules—the modules may be arranged in any suitable configuration. For example, the components for different modules may be interspersed with each other such that each module may be defined by the set of connected components that collectively perform a predetermined function. However, the components of each module may or may not be physically grouped within the cartridge. In some embodiments, multiple cartridges may be used to process a single cell product through transfer of the cell product from one cartridge to another cartridge of the same or different type and/or by splitting cell product into more cartridges and/or pooling multiple cell products into fewer cartridges.

As illustrated in FIG. 1B, the cartridge 114 may be configured to carry (e.g., house, secure, or enclose) a cell solution (e.g., cell suspension) for cell processing. Any number of cell processing steps may take place upon the cells within the cartridge. Accordingly, the cartridge 114 may include one or more of a bioreactor module 160, elutriation module 162, pump module 164, electroporation module 166, cell sorting module 168 (e.g., magnetic activated cell sorting (MACS)), and fluidic manifold 170. In some variations, the cartridge 114 may also include one or more of an additional sorting module (e.g., fluorescence activated cell sorting (FACS) module), an acoustic flow cell module, a microfluidic enrichment module, a transduction module, and/or the like.

The fluidic manifold 170) may be configured to transfer one or more fluids between one or more modules of the cartridge 114. For example, the fluidic manifold 168 may transfer a fluid (e.g., a cell solution) from the bioreactor module 150 to the cell sorting module 168. In another example, the fluidic manifold 168 may transfer a fluid from the cell sorting module 166 to any other module. In particular, the fluidic manifold 170 may transfer a fluid from the cell sorting module 168 to any other module after a magnetic cell sorting process has been performed. The fluidic manifold 170 may be configured to transfer the sorted cells (e.g., magnetically tagged cells) to a first module and the non-targeted cellular material to a second, different module.

The bioreactor module 160 may be configured to contain the cell solution. The bioreactor module 160 may include a mixing chamber, in which the cell solution may be mixed with one or more reagents. The one or more reagents may include, for example, transduction reagents (e.g., a lentiviral vector and/or a virus) or magnetic particles configured to couple to cells of a specific type.

The elutriation module 162 may be configured to perform an elutriation process whereby cellular material may be separated according to size, shape, and/or density.

The pump module 164 may be configured to pump fluid in one or more directions along at least one fluid path. For example, the pump module 169 may be configured to pump a fluid to or from one or more of the elutriation module 162, the bioreactor module 160, the fluidic manifold 170, the cell sorting module 168, and any other module within the cartridge 114.

The electroporation module 166 may be configured to facilitate intracellular delivery of macromolecules (i.e., transfection by electroporation).

The cell sorting module 168 may be a magnetic-activated cell sorting module comprising a flow cell and configured to separate target cells (e.g., magnetically-tagged cells) from non-target cellular material. In some variations, the flow cell of the cell sorting module 168 may be used for a transduction step of a cell processing workflow.

Various materials may be used to construct the cartridge (including the modules thereof) and the cartridge housing, including metal, plastic, rubber, and/or glass, or combinations thereof. The cartridge, its components, and its housing may be molded, machined, extruded, 3D printed, or any combination thereof. The cartridge may contain components that are commercially available (e.g., tubing, valves, fittings) and may be attached or integrated with custom components or devices. The housing of the cartridge may constitute an additional layer of enclosure that further protects the sterility of the cell product.

### 1.3 Controller

Referring back to FIG. 1A, the controller 120 may be configured to run the scheduler tool 130 described herein (e.g., via the processor 122). For example, a combination of software and hardware may be used to develop and update the scheduler tool. The software may include integrated software 140, such as MES to help run the scheduler tool 130. Hardware modules may include, for example, a general-purpose processor (or microprocessor or microcontroller), a field programmable gate array (FPGA), an application specific integrated circuit (ASIC), or the like. Software modules (executed on hardware) may be expressed in a variety of software languages (e.g., computer code), including MATLAB, C, C++, Java®, Python, Ruby, Visual Basic®, and/or other object-oriented, procedural, or other programming language and development tools. Examples of computer code may include, but are not limited to, micro-code or micro-instructions, machine instructions, such as produced by a compiler, code used to produce a web service, and files containing higher-level instructions that are executed by a computer using an interpreter. Additional nonlimiting examples of computer code may include control signals, encrypted code, and compressed code.

Further, the controller 120 may be communicably coupled to one or more components of the cell processing system 100, such as the workcell 110, the cartridge 114, and/or subcomponents thereof (e.g., the MHS 113, RVS 118, SLTS 115, SS 117, BPS 119, QCS 116, and/or one or more of the components 150). The memory 124 may store cell processing design plans, system instructions/commands, and the like. The processor 122 may execute the calculations and methods described herein and may be configured to receive and store data using the memory 124. The processor 122 may be any suitable processing device configured to run and/or execute a set of instructions or code and may include one or more data processors, image processors, graphics processing units, physics processing units, digital signal processors, and/or central processing units. In some variations, the controller 120 may include more than one controller. In some variations, the user interface 128 may allow an operator of the cell processing system 100 to view, modify, or otherwise interact with the cell manufacturing workflow (e.g., workflow considering all cartridges being processed within the system) or individual cell processing workflow for a single cartridge. In some variations, the user interface 128 may display recommendations for therapy modifications. The user interface 128 may be displayed on a suitable computing device which may include integrated software 140. In some variations, the display 126 may be configured to support the user interface 128. In some variations, the controller 120 may provide cell processing status notifications (e.g., digitally) via the user interface 128 (e.g., visual feedback, such as graphical and/or numerical feedback). In some variations, the controller 120 may be configured to automatically update one or more tools or processes (e.g., scheduler tool 130) using data collected during a cell processing workflow. The data may include, for example, an actual execution time for a given cell processing operation or a cell sample characteristic (e.g., cell count and/or cell viability) determined prior to or following a given cell processing operation.

### 1.3.1 Scheduler Tool

The scheduler tool 130 may generally be configured to schedule cell samples into an automated CPS (e.g., CPS 100) while managing system contention issues due to parallel processing such that the manufactured cell products meet all desired specifications (e.g., desired cell count and/or desired cell viability). The scheduler tool 130 may predict a cell manufacturing schedule that may organize multi-processing for all of a plurality of cartridges of the system. For example, the scheduler tool 130 may generate a loading schedule that reduces or eliminates overlapping periods of need for a single workcell component or subsystem based on aggregate estimations of system operations, manual operations, cell processing workflows (unique to each cell sample of the system), and intake sample and patient/donor parameters. The schedule may be based on a smallest amount of workflow contention that is acceptable to process. Workflow contention may arise from excessive or overlapping demand for a cell processing resource that cannot be met by the system. In some variations, high or excess contention may be identified for a system resource (e.g., one or more subsystems or components of the workcell 110) that is required for two or more cell samples workflows at the same (e.g., at least partially overlapping) periods of time. The scheduler tool 130 may be configured to identify and resolve (if possible) areas (or predicted areas) of high contention by ranking the two or more workflows involved and identifying a priority workflow of the two, as will be described in detail herein. In some variations, the scheduler tool 130 may be configured to suggest a procedure for manual resolution of contention if one or more cell samples are determined to have equally (or substantially equally) weighted resource contention.

The schedule (loading schedule) may be used (e.g., automatically by the CPS 100 and/or to inform an operator) to select an asynchronous loading time for one or more cell samples (e.g., cartridges carrying cell samples) and/or to reschedule/reconfigure one or more ongoing processing operations to resolve internal contention. In some variations, the schedule may be a plot and/or list of exclusion times for loading a proposed cartridge into a workcell. In some variations, the scheduler tool 130 may automatically update the predicted loading schedule and/or areas of contention based on real-time resource constraints of the system.

In some variations, the scheduler tool 130 may be an application configured to provide (e.g., visually and/or digitally display) cell processing information and/or configured to receive user input. For example, the scheduler tool 130 may provide one or both of a loading schedule for one or more cartridges and/or one or more contention statuses or action items (e.g., for an operator to assist in resolving contention). For example, the scheduler tool 130 may be accessed on one or both of the display 126 and the user interface 128. Additionally, or alternatively, the scheduler tool 130 may be accessed on a remote control, such as a mobile device, that is communicably coupled to the CPS 100 (e.g., to controller 120).

The scheduler tool 130 may be communicably coupled to one or more components of the CPS via the controller 120, such as to the MHS 113, RVS 118, SLTS 115, SS 117, BPS 119, and/or QCS 116. The scheduler tool 130 may be configured to receive and use real-time measurements of the system to inform a scheduling and/or contention resolution output. For example, QCS 116 may provide measurements and/or estimations of cell count and/or cell viability to the scheduler tool 130, which may use the measurements and/or estimations to update one or more workflow simulation inputs (and consequently, one or more outputs) of the scheduler tool 130.

### I. Scheduling a Cartridge for Loading

The scheduler tool 130 may utilize a combination of mechanisms to determine when a cartridge, such as cartridge 114, may be safely loaded into a workcell of a cell processing system, such as workcell 110 of CPS 100. To do so, the scheduler tool 130 may include a feedback control system for predicting a cartridge loading schedule based on environmental constraints of the CPS. The predictive schedule may organize parallel processing of all cartridges that are scheduled for processing within the system and all proposed cartridges to be scheduled for processing within the system (where each cartridge is associated with a unique cell processing workflow). Additionally, the scheduler may compensate for load time and/or process variability by padding the input data to limit potential downstream effects of loading and/or process variability.

The scheduler tool 130 may include one or more inputs for determining a cartridge loading schedule.

Nonlimiting examples of inputs for scheduling cartridge loading, which are discussed in detail herein, may include one or more of: (1) configuration of the cell processing system. (2) configuration of a load window (may define an acceptable variation of the nominal time for the cartridge to be loaded into the system), (3) scheduled cartridges and associated respective design plans and simulated workflows. (4) proposed (e.g., to-be-scheduled) cartridge and associated design plan and simulated workflow, and (5) intake sample parameters. In some variations, the system may determine a debugging configuration.

The intake or initial sample parameters may include data such as, but not limited to, cell type (e.g., autogenic versus allogenic), sample collection time, sample transport time (e.g., to a facility running a CPS such as CPS 100), sample arrival time (e.g., at a facility running a CPS such as CPS 100), sample temperature (e.g., frozen vs. thawed), patient/donor age, patient/donor sex, and/or additional patient/donor information (e.g., medical history). Estimated initial parameters such as expected recovery from cell separation and/or expected recovery time may also inform the process design for a cell sample. The initial sample parameters may be manually entered into the system (e.g., via user interface 128).

In some variations, the cell processing system may be configured to automatically generate one or more of the inputs (e.g., via processor 122), such each of aforementioned inputs 1-4. In some variations, a plurality of cartridges (e.g., at least one, one or more, or two or more cartridges) may be analyzed simultaneously by the scheduler tool 130 to determine when each of the cartridges may be entered into the system.

The scheduler tool 130 may be configured to automatically determine one or more updated inputs at any time during a manufacturing process (e.g., before and after each operation for each cartridge within the system). For example, the cell processing system configuration may be updated using real-time resource constraints, as one or more resources (e.g., one or more reagents, available BPSs, etc.) may be diminished as the manufacturing process continues. As another example, the scheduler tool 130 may be configured calculate an actual (e.g., realized) load window for one or more cartridges, and use the calculated load window to reforecast the manufacturing plan, as described in detail herein, or for operator or process feedback compensation.

Using one or more of the inputs described herein, the scheduler tool 130 may generate one or more outputs related to safely loading a proposed cartridge into the scheduler tool. The scheduler tool 130 may provide the outputs visually, such as graphically and/or numerically, via the display 126 and/or the user interface 128. In some variations, an operator may edit one or more outputs to override decisions/suggestions made by the scheduler tool 130. In some variations, the scheduler tool 130 may prompt an operator to accept or reject a loading plan and/or contention resolution plan. Nonlimiting examples of such outputs may include one or more of: (1) a schedule of unavailable (exclusion) times for loading. (2) a schedule of available times for loading. (3) a proposed time for loading the cartridge, and (4) a true/false result for one or more proposed cartridge loading times (e.g., indicating whether the proposed entry time is acceptable or not). A schedule output may additionally include estimated times for one or more cartridges to be ejected from the system (e.g., via the feedthrough 111).

In some variations, the scheduler tool 130 may be configured to reforecast one or more outputs periodically during the manufacturing process. For example, the scheduler tool 130 may reforecast a cartridge loading schedule before and after each cell processing operation for each cartridge within the system (e.g., using updated data collected during the preceding processing event).

Additionally, or alternatively, the scheduler tool 130 may be configured to provide a schedule for loading fluid devices (e.g., fluid device 155), such as for loading SLTDs within the RVS (e.g., RVS 118).

In some variations, the scheduler tool 130 may additionally be configured to output past, real-time, and/or predicted system statistics. These statistics may include, for example, a summary of robot motions (e.g., for robot 112 of the MHS 113) and/or SLTD statistics.

The scheduler tool 130 may include one or more predictive models. Nonlimiting examples of such models may include one or more of a neural network (e.g., CNN), decision tree, a linear regression, a classification model, a random forest, and the like.

i. Input: Cell Processing System Configuration

The cell processing system configuration may be defined by one or more of a type and/or quantity of workcell instruments or subsystems, fixed timing constraints associated with the subsystems, a type and/or quantity of reagents stored within the system (e.g., in RVS 118), and the like. A fixed timing constraint may refer to timing constraint that is not workflow specific. That is, a fixed timing constraint may a timing constraint that is inherent to the operation of the CPS subsystem or component. For example, the system configuration may have a fixed timing constraint for fluid transfer among components (e.g., via the SLTS 115), cartridge and/or fluid device transfer among the workcell (e.g., via MHS 113), sterilization of a cartridge and/or fluid device (e.g., via the SS 117), etc. In some variations, the system configuration may include identifying one or more cartridges that are currently being processed by the system. Moreover, the system configuration may include a load configuration such as a number of hours, days, weeks, and/or months that the system may operate, and/or a number and/or length of operator shifts for operating the system.

Thus, the cell processing system configuration may define the real-time capabilities of a CPS based on resource availability and fixed timing constraints of the system. Generally, with more internal resources available, more cartridges may be entered into the system, which may increase throughput. However, in some cases, adding instruments to the system may not impact a critical processing path (e.g., processing operation(s) required by multiple cartridges for a shared time period) and may therefore not increase system throughput. Similarly, in some cases, adding instruments to the system to achieve internal redundancy (fault robustness) may not result in higher throughput.

ii. Input: Load Window Configuration

While the scheduler tool 130 may be configured to identify an ideal load time for a cartridge (e.g., at a specified time of day, to the minute), in practice, it may be unlikely that an operator enters the cartridge into the system at the exact time specified. Accordingly, a load window configuration may define an acceptable margin of error around the nominal time identified (e.g., a specific time of day such as “14:00”) for a cartridge to enter the workcell. In particular, the load window may include symmetrical margins of error around a nominal identified time for loading. For example, a load window of 2 hours may allow an operator to enter a proposed cartridge into the workcell at some point within the hour before the identified loading time or within the hour after the identified loading time. That is, a nominal loading time of 12:00 with a 2-hour loading window would allow the operator to load the cartridge into the workcell at any time between 11:00 and 13:00.

Alternatively, in some variations, the load window may have asymmetrical margins around the nominal identified loading time. For example, the load window may be a time period that is only before or only after the identified loading time.

The load window may be on the order of seconds, minutes, or hours. In some variations, the load window may be between 30 minutes and about 3 hours, such as between about 45 minutes and about 2.5 hours, between about 1 hour and about 2 hours, or between about 1.25 hours and about 1.75 hours. In some variations, the load window may be below 5 minutes, about 5 minutes, about 10 minutes, between about 5 minutes and about 10 minutes, about 15 minutes, about 30) minutes, about 45 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, or greater than 5 hours.

Generally, a large load window (e.g., on the order of several hours, such as about 2, 3, 4, or 5 hours) may indicate that the scheduler tool 130 is not controlling or improving an overall cell process plan, and consequently limiting throughput by limiting a number of cartridges that may be loaded into the workcell. Oppositely, a small load window (e.g., on the order of several seconds or several minutes, such as about 2, 5, 10), or 30 minutes) may increase a likelihood of an operator missing the load window and having to reschedule the proposed cartridge, which may also reduce throughput. Ideally, the load window configuration input may allow for variability in cell sample (and/or cartridge) preparation while constraining a potential for reduced cell product throughput due to the allowed variability in sample preparation.

In some variations, load time variation (actual load time versus scheduler-identified load time) may be compensated by the scheduler tool 130 (e.g., using feedback compensation) to limit an impact of the load time on overall process variability (e.g., to reduce overall process variability).

iii. Input: Scheduled Cartridges and Simulated Workflows

The scheduler tool 130 may evaluate scheduled (e.g., previously scheduled) cartridge of the workcell to determine when a proposed cartridge may be added to the system. All of the scheduled cartridges and their respective workflows (process design plans) may additionally be inputs for the scheduling tool. For example, the scheduler tool 130 may include one or more predictive models for simulating a workflow for each of a plurality of currently scheduled cartridges. The one or more predictive models may be trained on results and/or estimates of past cell processing workflows and/or comparable manual cell processing operations. For each scheduled cartridge, the one or more predictive models may receive data including, but not limited to: cell sample collection time, cell sample arrival time (e.g., at a facility running a CPS such as CPS 100), initial cell count, expected recovery from cell separation, expected recovery time, cell sample temperature (e.g., frozen vs. thawed), patient/donor age, and patient/donor sex. Next, the one or more predictive models may simulate a workflow (e.g., including a schedule of cell processing operations and associated time estimates) for the given scheduled cartridge.

In some variations, the scheduler tool 130 may account for workflow variability (e.g., due to loading and/or process variation) by padding the data for one or more of the simulated workflows. Padding scheduled cartridge data may involve the following steps: (1) predicting a nominal workflow based on loading at an ideal/planned time with no process variation. (2) padding the simulated nominal workflow to account for potential variability within the load window, and (3) padding the simulated nominal workflow to account for a minimum and maximum variability of the workflow operations. This data padding may be performed for one or more, or all of, the simulated workflows for a plurality of scheduled cartridges.

Padding of simulation data may take on different forms, such as padding with or without variation compensation. In some variations, workflow variation compensation may allow the load window variation to be compensated out during a first extended (e.g., hours-long or days-long) cell processing operation. That is, the load window variation may be compensated for during a processing operation such as expansion, which may take between, for example, 4, 5, 6, 7, 8, 9, 10, or more than 10 days. Accordingly, load window variation may only impact early processing operations (e.g., within a first day of a cell processing workflow) due to the cell sample undergoing an extended processing operation, during which the variation may be compensated for. Process variability compensation may be performed similarly to load variability compensation but also may occur throughout the entire workflow.

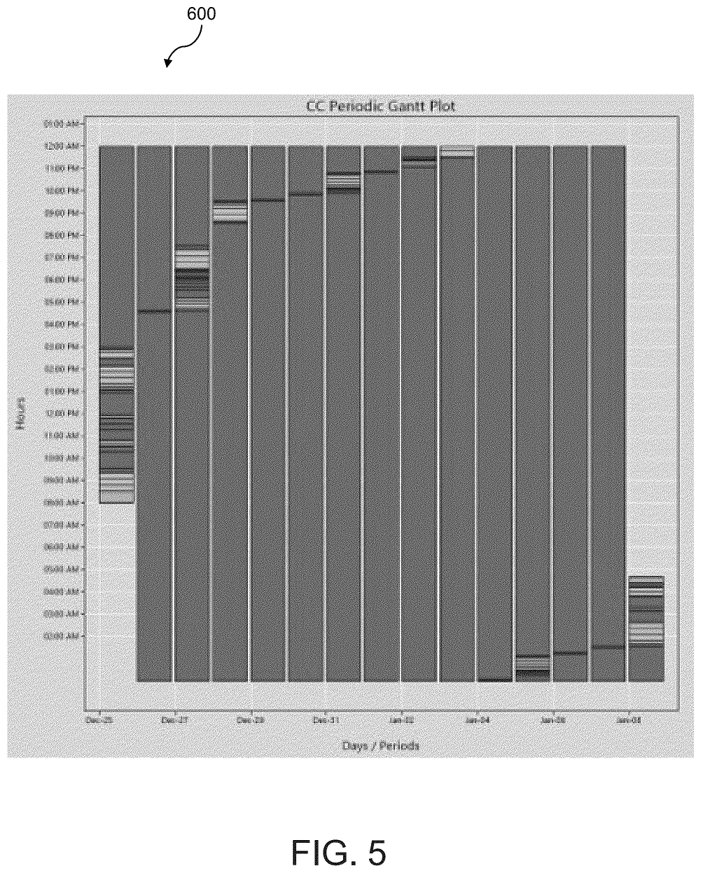

An exemplary simulated workflow for a cartridge is described in Example 1 herein with reference to FIG. 5

iv. Input: Proposed Cartridge Simulated Workflow

The scheduler tool 130 may also include one or more predictive models for simulating an associated workflow for a proposed cartridge. The one or more predictive models may be trained on results and/or estimates of past cell processing workflows and/or comparable manual cell processing operations. As with the scheduled cartridge simulated workflow input, the workflow for the proposed cartridge may be modeled (e.g., via the one or more predictive models) based on one or more intake parameters and the process design plan for the proposed cartridge. For example, the one or more predictive models may receive data including, but not limited to: cell sample collection time, cell sample arrival time (e.g., at a facility running a CPS such as CPS 100), initial cell count, expected recovery from cell separation, expected recovery time, cell sample temperature (e.g., frozen vs. thawed), patient/donor age, and patient/donor sex. Next, the one or more predictive models may simulate a workflow (e.g., including a schedule of cell processing operations and associated time estimates) for the proposed cartridge.

Additionally, as described above, the proposed cartridge workflow data may be padded to account for loading and/or process variation.

Generally, cartridges having shorter process designs (e.g., including fewer and/or quicker cell processing operations compared to an average workflow) may have fewer contention issues than cartridges with longer process designs (e.g., including more and/or longer cell processing operations compared to a workflow for a cell sample of another cartridge).

v. Output: Loading Schedule

The scheduler tool 130 may use one or more of the inputs described herein to determine a cartridge loading schedule for a proposed cartridge. For example, the scheduler tool 130 may be identify and output one or more times (e.g., time periods, such as a set of time periods) that are unavailable (or, alternatively, available) for loading a proposed cartridge (or for loading one or more of a plurality of proposed cartridges) into a workcell. In some variations, the scheduler tool 130 may include one or more predictive models configured to provide the output, such as one or more neural networks. In some variations, the output may include more than one schedule (e.g., a plurality of schedules), such as a schedule of unavailable loading times and a schedule of available loading times for a cartridge.

For example, the output may include a list or schedule of exclusion times for loading a proposed cartridge. The exclusion times may be one or more time periods during which a proposed cartridge cannot be loaded into a workcell due to one or more subsystems operating at capacity and/or one or more subsystems having contention. For example, a set of exclusion times (e.g., a closed set of exclusion times) may include times during which the scheduled cartridges are predicted to be loaded and/or times during which cell processing components or subsystems (e.g., the SLTS 115 and/or the BPS 119) are predicted to have high contention considering the requirements of the scheduled workflows. In some variations, each excluded time period of the set of exclusion times may be a block of time beginning and ending at about the same times that a predicted contentious event (e.g., loading a scheduled cartridge) begins and ends.

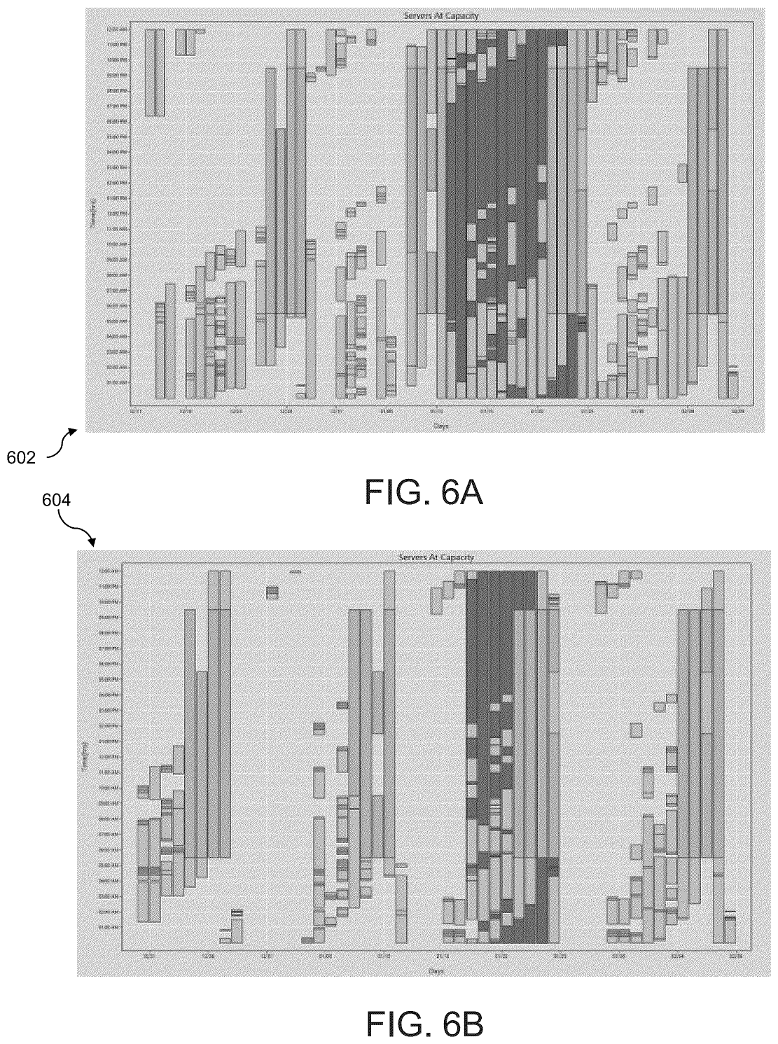

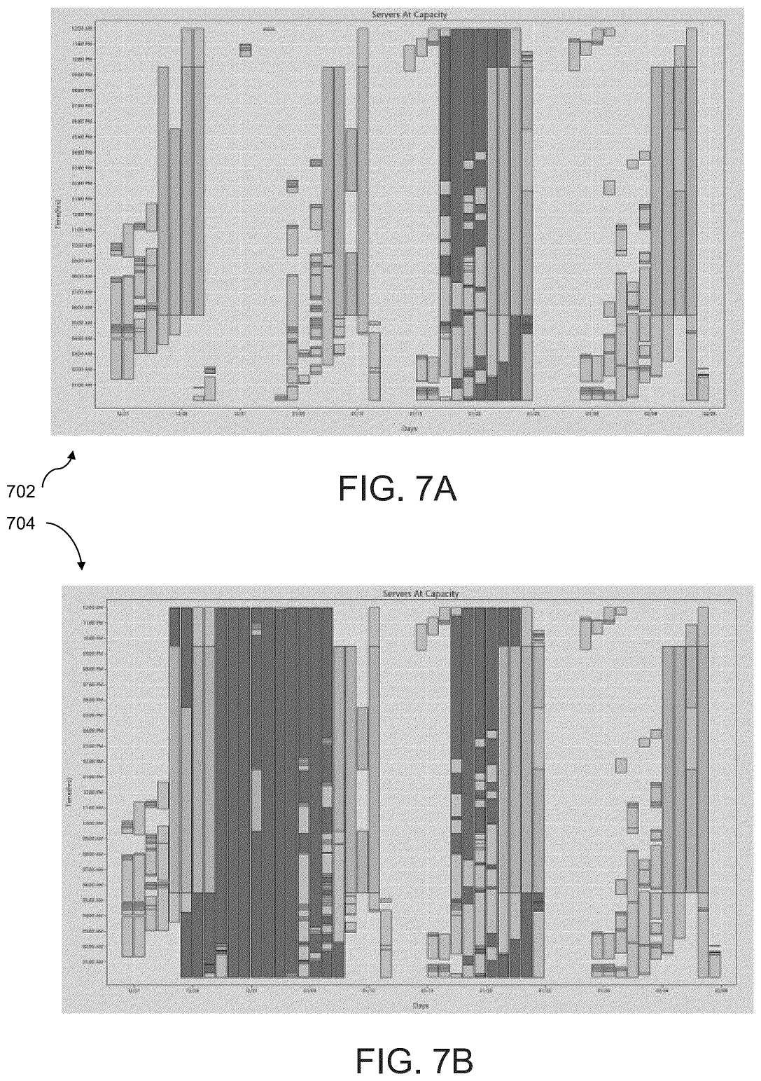

The scheduler tool 130 may output a schedule of exclusion times visually, such as graphically and/or numerically, via a display and/or user interface (e.g., display 126, user interface 128). In some variations, the schedule may be color coded to support interpretation by an operator. In some variations, the schedule of exclusion times may additionally identify a type of exclusion time. For example, the schedule may identify exclusion times covering load windows for scheduled cartridges differently (e.g., using different colors) than exclusion times covering periods of high contention. In some variations, the schedule may identify exclusion times covering periods of high contention for a first subsystem of the workcell (e.g., the SLTS 115) differently than exclusion times covering periods of high contention for a second subsystem of the workcell (e.g., the BPS 119), and may function similarly to identify exclusion times covering periods of high contention for any number of subsystems or components of the workcell. In some variations, the schedule may identify periods of acceptable contention (e.g., contention where two or more workflows may require a same resource at close, but non-overlapping time periods) for the operator's reference. Exemplary schedules of unavailable loading times for a cartridge are described in Examples 2 and 3 herein with reference to FIGS. 6A-6B and FIGS. 7A-7B, respectively.

Oppositely, in some variations, the output may include a list or schedule of available times for loading a proposed cartridge. The available times may be one or more time periods during which a proposed cartridge can be loaded into a workcell. In some variations, the available times may include a set of time periods beginning at time zero (e.g., beginning prior to loading a first scheduled cartridge) and running indefinitely. Accordingly, a list of available times for loading a proposed cartridge may be less constrained than a set of exclusion times for loading the proposed cartridge.

In some variations, the output may include one or more recommended times for loading a proposed cartridge. Additionally, in some variations, the output may include a true/false result associated with at least one of the one or more recommended times for loading the proposed cartridge. The true/false result may indicate whether the proposed entry time is acceptable or not).

In some variations, the scheduler tool 130 may be configured to solve a “job shop” constraint satisfaction problem (CSP) considering all scheduled and proposed cartridges. The CSP may define a number of cell processing operations to be completed using a specific number and sequence of components of the workcell(s) processing the cartridges. Scheduling with CSP may allow for efficient and flexible modeling of constraints and may also handle complex and dynamic constraints which may arise during cell therapy manufacturing.

The scheduler tool 130 may solve the CSP for scheduling an automated CPS by modeling the cell therapy manufacturing process as a set of jobs and the instruments used to perform the jobs as the resources required to complete them. The output may include an optimal cartridge loading schedule that satisfies constraints including one or more of (e.g., all of) instrument availability, operation precedence, operation criticality, maximum batch (e.g., cell sample) cell count, and maximum batch incubation time. The optimal schedule output may optimize resource utilization within the workcell(s) and reduce cell processing cycle costs and time are reduced (e.g., meet processing deadlines).

### II. Managing System Contention

The scheduler tool 130 may be used to monitor progress of the manufacturing process and to make real-time adjustments to ensure that all cell products meet required specifications and quality standards (e.g., meet a predetermined threshold for acceptable cell count and/or cell viability). For example, system resource requirements and workflows of cell samples being processed may be evaluated continuously (e.g., based on timing of cell processing events) to recalculate areas of contention and/or to reforecast areas predicted contention. In some variations, the scheduler tool 130 may determine contention for each resource/instrument of the system by considering the processing requirements for each cartridge being processed before and after every cell processing operation is executed within the system.

An amount of contention may be determined from a number of cartridges (or associated workflows) that require a resource of the system, or at least a portion of the resource, at a same time. In some variations, the system contention may be determined from a ratio of cartridges (or associated workflows) to CPS subsystems and/or components needed during a given time period. For example, the scheduler tool 130 may compare a number of contentious cartridges and/or a ratio of contention to a predetermined threshold for acceptable contention in order to quantify an identified the subsystems and/or component contention. Generally, the lower the contention ratio, the less contention there may be in the system. Additionally, a length of overlap time between, for example, two cartridges requiring a same system resource may determine an amount of contention that exists. For example, two cartridges requiring the same SLTS for 5 seconds of overlap time may cause less contention than two cartridges requiring the same SLTS for 30 minutes of overlap time. Further, the amount of contention may depend on a dwell time for a current processing operation and/or an execution time for a subsequent processing operation for each of the cartridges in contention for a given instrument. The dwell time and/or execution time for each cartridge may be used to determine a weight of instrument contention for the cartridge, as discussed in detail herein.

In some variations, the scheduler tool 130 may be configured to determine and provide a confidence estimate for one or more calculated areas of contention. The scheduler tool 130 and/or an operator may be able to configure and/or reconfigure a confidence threshold for comparing to one or more confidence estimates determined by the scheduler tool 130.

In some variations, the scheduler tool 130 may be configured to recalculate contention of the system with updated data (e.g., collected during a preceding processing operations) for one or more cartridges of the system (e.g., cartridges being processed, scheduled, and proposed) to determine if areas of contention may arise due to a difference between the actual and simulated results. In some variations, the scheduler 130 may be configured to compare the calculated operation execution time and the corresponding simulated operation execution time to determine if there is a significant difference (e.g., about 3%, 4%, 5%, 6%, 7%, 8%, 9%, 10%, 15%, 20%, or greater than about 5% difference) between the two. Generally, the difference between calculated and simulated event times may be minimal when instrument contention is reduced or avoided via scheduling by the scheduler tool 130. In such cases, contention and associated deviation from simulated (ideal) results may be limited to small amounts (e.g., within about 5%, about 10%, about 15% or about 20% of the simulated results) primarily as a function of robotic interactions timing (e.g., competition for the RVS 118 and/or the SLTS 115) within the workcell. As such, a deviation that is less than a typical process variation (e.g., within about 15% of the predicted results) may have minimal impact on the reliability of the outputs of the scheduler tool 130.

When high or excess contention (e.g., time periods of required resource overlap and/or numerous cartridges requiring one or more resources) is identified (e.g., during contention recalculation before or after a given cell processing operation), the scheduler tool may suggest (e.g., to an operator via display 126 and/or user interface 128) or automatically implement resolutions for areas of high contention based on priority constraints of the cell samples. For example, resolving contention may involve rescheduling/reconfiguring a workflow of one or more cartridges (e.g., extending or reducing a time period for a cartridge to dwell within a subsystem).

The priority constraints may be evaluated based on dwell times for current or completed cell processing operations and execution times for subsequent cell processing operations. Comparing the priority constraints of two or more cell samples may include determining a weight of instrument contention for each of the cell samples, where a cell sample having a larger weight of contention for the given instrument may be the cell sample with the highest priority constraints. The scheduler tool 130 may use the comparison to prioritize (or reprioritize) one or more cell samples requiring a same resource ensure high quality cell products that meet the conditions of the manufacturing process. For instance, when two different cell samples require an SLTS, but one batch is constrained by the need for a viral vector (which must be used within 30 minutes of thawing), it may be assigned priority to use the resource. Cell sample operations that are determined to have equally weighted resource contention may cause operators to be alerted with a recommended possible resolution.

The dwell time may be a maximum amount of time allowed between a completed processing operation and a subsequent processing operation that does not affect cell product quality. The execution time may include minimum and maximum execution times, which may be an estimated time to complete a subsequent processing operation with and without padding the time estimate (e.g., to account for process variability), respectively. The scheduler tool 130 may compare the dwell time for one or more cell samples to the minimum and maximum execution times for one or more different cell samples to determine which sample should be prioritized and proceed to use the contentious resource.

For example, when minimum and maximum execution times for a first cell sample are both less than a dwell time for a second cell sample, the first cell sample may be assigned priority to proceed with its subsequent processing step (as the maximum execution time accounts for variance in the actual execution time of the subsequent processing operation).

However, when the minimum execution time for the first cell sample is greater than the dwell time for the second cell sample, the scheduler tool 130 may proceed to evaluate the workflow of the first cell sample to determine it can be delayed. If the first cell sample can be delayed, then the second cell sample may be assigned priority to proceed with its subsequent processing step. If the first cell sample cannot be delayed (e.g., is dependent on use of a viral or lysis vector), then the scheduler tool 130 may determine that the first and second cell samples have equally weighted resource contention. The scheduler 130 may then notify an operator (e.g., via the display 26 and/or the user interface 28) that contention between the first and second cell samples cannot be resolved. The operator may then manually assign priority to one of the first and second cell samples, may move one or both of the first or second cell samples to a different workcell (which can meet the demand for the in-demand resource), and/or or may manually perform the subsequent processing operation for one or both of the first and second cell samples.

Similarly, when the maximum execution time for the first cell sample is equal to (or substantially equal to) the dwell time for the second cell sample, the scheduler tool 130 may determine that the first and second cell samples have equally weighted resource contention. The scheduler 130 may notify an operator (e.g., via the display 26 and/or the user interface 28) that contention between the first and second cell samples is not resolved. The operator may then determine how to resolve the contention.

Moreover, when the maximum execution time for the first cell sample is greater than the dwell time for the second cell sample, the second cell sample may be assigned priority to proceed with its subsequent processing step.

In some variations, the scheduler tool 130 may include a confidence model, which may be a model configured to provide a confidence estimate associated with one or more dwell times, minimum execution times, and/or maximum execution times determined by the scheduler tool 130. The scheduler tool 130 may provide such estimates graphically (e.g., confidence intervals) and/or numerically via the display 126 and/or user interface 128. For example, the scheduler tool 130 may be configured to provide a confidence estimate (e.g., confidence interval) associated with minimum and maximum execution times for a given cell sample. One or both of the scheduler tool 130 and the operator may interpret the confidence estimates to determine how risky it may be to, for example, assign priority to a first cell sample having a minimum execution time that is about equal to than a dwell time for a second cell sample. In some variations, the scheduler tool 130 may be configured to automatically assign priority to the second cell sample to proceed to its subsequent processing operation when the confidence estimate for the minimum execution time for the first cell sample is less than about 95% or less than about 90%. As it follows, in such variations, the scheduler tool 130 may be configured to automatically assign priority to the second cell sample to proceed to its subsequent processing operation when the confidence estimate for the minimum execution time for the first cell sample is greater than about 90% or greater than about 95%.

FIG. 2 is a flow chart illustrating an exemplary strategy for the scheduler tool 130 to evaluate and resolve contention between two cell samples. To carry out both procedures 202, 204, the scheduler tool may be configured to calculate the minimum (procedure 202) or maximum (procedure 204) execution time for a subsequent operation of a first cell sample, operation A, and the dwell time for a current (e.g., real-time) operation of a second cell sample, operation B. While the procedures 202, 204 outline strategies for comparing the dwell time for operation B to a minimum or maximum execution time for operation A, in some variations, the scheduler tool 130 may be configured to compare the dwell time for operation B to both the minimum and maximum execution times for operation A, or to compare any combination of operation B dwell, minimum execution, and maximum execution times to any combination of operation A dwell, minimum execution, and maximum execution times.

To perform procedure 202, the scheduler tool 130 may compare the minimum execution time for operation A to the dwell time for operation B. If the minimum execution time for operation A is within (e.g., less than, or less than or equal to) the dwell time for operation B, the scheduler tool may assign priority to the first cell sample to be processed with operation A. If the minimum execution time for operation A is not within (e.g., greater than, or greater than or equal to) the dwell time for operation B, the scheduler tool may further evaluate operation A to determine if the first cell sample can dwell. For example, if operation A includes introducing a viral or lysis vector to the first cell sample, then operation A may not be delayed beyond 30 minutes of defrosting the viral vector or within 45 minutes of defrosting the lysis vector. Accordingly, if operation A may not be delayed, then the scheduler tool 130 may notify an operator that the contention between the first and second cell samples cannot be resolved. Otherwise, if operation A can be delayed, the scheduler tool 130 may assign priority to the second cell sample to be processed with operation B. In executing the procedure 202, the scheduler tool 130 may be configured to calculate a confidence estimate for the minimum execution time of operation A. If the confidence estimate for the minimum execution time is below a predetermined threshold, such as below 90% (as shown in FIG. 5), then the scheduler tool 130 may be configured to assign priority to the first cell sample to be processed with operation A.

To execute procedure 204, the scheduler tool 130 may compare the maximum execution time for operation A to the dwell time for operation B. If the maximum execution time for operation A is less than (or less than or equal to) the dwell time for operation B, then the scheduler tool 130 may be configured to assign priority to the first cell sample to be processed with operation A. If the maximum execution time for operation A is greater than (or greater than or equal to) the dwell time for operation B, then the scheduler tool 130 may be configured to assign priority to the second cell sample to be processed with operation B. If the maximum execution time for the operation A is determined to be equal to (or substantially equal to) the dwell time for the second cell sample, the scheduler tool 130 may determine that the first and second cell samples have equally weighted resource contention. The scheduler 130 may then notify an operator that contention between the first and second cell samples is not resolved. The operator may then determine how to resolve the contention.

A practical example for which the scheduler tool 130 may use one or both of procedures 202, 204 for resolving contention between the first and second cell samples may be in the context of competition for an SLTS (e.g., SLTS 115). In particular, in some cases, there may be only one SLTS available, operation A may include a lysis buffer transfer in a first SLTD via the SLTS to the first sample in a first BPS, and operation B may include a wash buffer transfer in a second SLTD via the SLTS two SLTSs to the second sample in a second BPS. Accordingly, the area of contention may include the single available SLTS. Operation A may have a minimum execution time of about 20 minutes, a maximum execution time of about 45 minutes, and a dwell time of about 30 minutes. Operation B may have a minimum execution time of about 30 minutes, a maximum execution time of about 45 minutes, and a dwell time of about 10 minutes. Accordingly, the scheduler tool 130 may determine, via procedure 204, that operation B, with a smaller dwell time than maximum execution time of operation A, may be assigned priority. In some cases, determining priority between operations A and B may depend on a lysis vector necessary for a remaining portion of operation A. For example, if a lysis vector for operation A is not defrosted and/or was just defrosted (to be used within 45 minutes), then operation B, with a smaller dwell time minimum and maximum execution time of operation A, may be assigned priority. However, by procedure 202, the scheduler tool 130 may evaluate operation A to determine if it can be delayed. If the lysis vector is defrosted and must be used within about 30-45 minutes (the minimum execution time of operation B), then, the scheduler tool 130 may notify an operation to aid in resolving the contention.

In variations where more than two cell samples may be involved (or predicted to be involved) in contention for an instrument, the scheduler tool 130 may resolve the contention (e.g., determine, if possible, which cell sample should be prioritized to proceed to a subsequent processing operation) by: (1) comparing the priority constraints of each of the more than two cell samples (as described above with respect to the first and second cell samples), and (2) stack ranking each of the two or more cell samples based on their respective dwell, minimum execution, and maximum execution times. If the two or more samples are determined to have equally weighted resource contention (e.g., cannot be ranked), then the scheduler tool 130 may indicate (e.g., via the display 26 and/or the user interface 28) that the contention cannot be resolved. To resolve the contention, the operator may then manually assign priority to the two or more samples, may move one or more of the cell samples to a different workcell (which can meet the demand for the in-demand resource), or may manually perform the subsequent processing operation for one or more of the cell samples.

In some cases, high contention may be resolved by simply adding an in-demand resource, such as a fluid, into the system during active manufacturing. For example, if two or more cartridges require a total amount of reagent that is greater than the amount of reagent currently stored in the system (e.g., in the RVS 118), then the scheduler tool 130 may configured to notify the operator to add into the system (e.g., via an access point of the RVS 118) an amount of reagent equal to or greater than the total amount required by the two or more cartridges in order to resolve the contention.

### 2. Methods for Cell Processing

Generally, the systems described herein may perform one or more methods for cell processing, including one or more methods for scheduling and/or managing the processing of multiple cartridges. In some variations, the one or more methods may be computer-implemented. For example, the methods described herein may be executed via a controller such as controller 120 of FIG. 1A. The methods herein may generally include providing a notification (e.g., visual, audio, haptic, etc.) to an operator of the cell processing system (e.g., via a display such as display 126 and/or a user interface such as user interface 128) indicating (1) an output of the scheduler tool 130 (e.g., a loading schedule for proposed cartridges), (2) a contention status and/or indicating changes being made to a workflow of one or more cartridges within the system in order to resolve, limit, or avoid contention among the cartridges, or (3) a manual decision/action required to proceed with the manufacturing plan (e.g., operator action required to resolve contention). In some variations, the operator may optionally provide feedback to the CPS before, during, or after one or more steps of the methods herein.

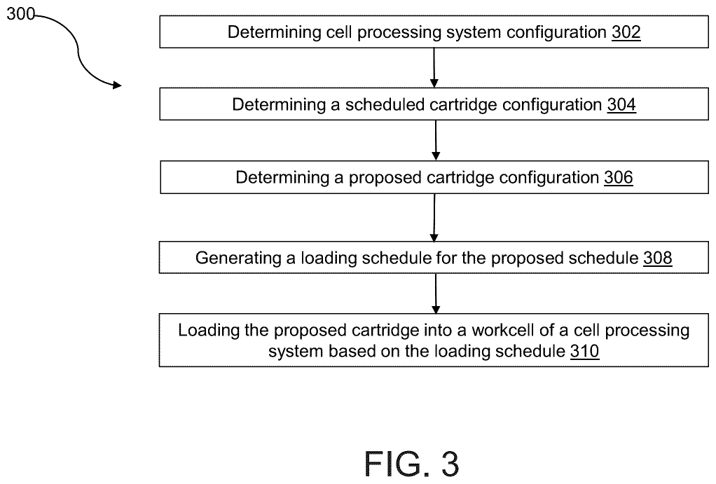

FIG. 3 provides a flowchart of an illustrative variation of a method 300 for cell processing. In particular, the method 300 may be for scheduling a proposed cartridge for cell processing. In some variations, a tool, such as the scheduler tool 130 of FIG. 1A, may be configured to execute (e.g., automatically execute) the method 300.

First, the method 300 may include determining 302 a cell processing system configuration. The CPS configuration may define the real-time and/or predicted capabilities of a CPS based on resource availability and fixed timing constraints of the system. Resources may include a resource within the RVS (e.g., RVS 188), such as one or more of culture media, buffer, cytokines, proteins, enzymes, polynucleotides, transfection reagents, non-viral vectors, viral vectors, antibiotics, nutrients, cryoprotectants, solvents, cellular materials, and pharmaceutically acceptable excipients. Fixed timing constraints of the system may include, for example, a time required for the robot of the MHS (e.g., robot 112 of MHS 113) to move a cartridge from one subsystem to another (e.g., from the feedthrough 111 to the BPS 119). As another example, a fixed timing constraint may include a time required for the SS (e.g., SS 117) to perform a sterilization procedure for a cartridge.

Next, the method 300 may include determining 304 a scheduled cartridge configuration, such as determining at least one, one or more, or a plurality of scheduled cartridge configurations. The scheduled cartridges may be cell processing cartridges (e.g., each carrying a cell sample and configured to interface with the CPS, such as cartridge 114) that have been previously scheduled for processing within the CPS. That is, the loading times (for loading the cartridges into the CPS) for the scheduled cartridges may have already been defined prior to execution of the method 300. The determining 304 may include identifying a first scheduled cartridge and/or a process design plan for a first scheduled cartridge, and may additionally include receiving one or more sample intake parameters for the cell sample carried by the first scheduled cartridge. Nonlimiting examples of sample intake parameters may include one or more of: a sample cell type, a sample collection time, a transport time for the sample, a processing facility arrival time for the sample, a sample temperature, patient age, patient sex, donor age, and donor sex. The determining 304 may involve modeling (e.g., via one or more predictive models) a workflow for the first scheduled cartridge based on the process design plan (workflow) and the one or more sample intake parameters. In some variations, the simulated workflow may additionally or alternatively be based on real-time data (e.g., updated cell processing results). In some variations, the one or more scheduled cartridges may be undergoing processing in real-time, and any real-time estimations or measurements associated with the cartridges (e.g., realized cell processing operation execution times, cell counts, cell viability estimations) may be used to determine and/or reforecast their associated scheduled cartridge configurations. In some variations, one or more scheduled cartridge configurations may be generated for visualization and/or editing by an operator (e.g., via a user interface such as user interface 128). The determining 304 may occur any number of times for each of a plurality of scheduled cartridges.

Next, the method 300 may include determining 306 a proposed cartridge configuration. The proposed cartridge may be a cell processing cartridge that is not yet scheduled for processing within the CPS. The determining 306 may include designing and/or identifying a process design plan for the proposed cartridge, and may additionally include receiving one or more sample intake parameters for the cell sample carried by the proposed cartridge (as listed above with respect to the determining 304). The determining 306 may involve modeling (e.g., via one or more predictive models) a workflow for the proposed cartridge based on the process design plan (workflow) and the one or more sample intake parameters. Additionally, or alternatively, the proposed cartridge configuration may include a loading configuration that defines an acceptable time period within which an operator may load the proposed cartridge into the CPS. In some variations, the proposed cartridge configuration (e.g., the simulated workflow and/or the loading configuration) may be generated for visualization and/or editing by an operator (e.g., via a user interface such as user interface 128). In some variations, the determining 306 may be carried out for each of a plurality of proposed cartridges.

Further, the method 300 may include generating 308 (providing) a loading schedule for loading the proposed cartridge. The schedule may be generated based on the system configuration and the schedule and proposed cartridge configurations. The generating 308 may include displaying the schedule (e.g., in graphical and/or numerical form) via a display (e.g., display 126) and/or a user interface (e.g., user interface 128). The schedule may provide one or more exclusion times for the proposed cartridge, which may be periods of time during which the cartridge may not be loaded into the CPS. Additionally, or alternatively, the schedule may provide one or more available times for loading the proposed cartridge into the system. Thus, the generating 308 may include predicting system contention (e.g., cartridge contention for one or more subsystems or instruments of the system) caused by loading the proposed cartridge, and reducing or resolving the predicted contention to create the schedule. Accordingly, the schedule may be an optimal schedule for loading the proposed cartridge which minimizes system contention and therefore sample viability risk. Managing system contention is discussed in more detail with respect to method 400 of FIG. 4, which may be used in conjunction with the method 300.

Referring again to FIG. 3, the method 300 may finally include loading 310 the proposed cartridge into the CPS (e.g., into the workcell 110) based on the loading schedule. That is, an operator may be directed by the loading schedule to load the proposed cartridge into the system. For example, an operator may chose a loading time that does not overlap with any of one or more unavailable loading times provided by a loading schedule in which to load the proposed cartridge into the system. In some variations, the loading schedule may identify an ideal time for loading the proposed cartridge, and the operator may load the proposed cartridge at this time (or within a load window based on this time).

One example of a method for cell processing, including scheduling a proposed cartridge for cell processing, may first include determining a system configuration of a cell processing system configured to process a plurality of cartridges in parallel. Each of the plurality of cartridges may be configured to carry a cell sample and to be loaded into a workcell of the cell processing system. Next, the method may include determining a cartridge configuration for each of the plurality of cartridges and providing a loading schedule for loading a first cartridge of the plurality of cartridges into the workcell. The loading schedule may be based on the system configuration and the configuration for each of the plurality of cartridges.

Moreover. FIG. 4 provides a flowchart of an illustrative variation of a method 400 for cell processing. In particular, the method 400 may be for monitoring and/or managing contention within a cell processing system configured for parallel processing (e.g., CPS 100). In some variations, a tool, such as the scheduler tool 130 of FIG. 1A, may be configured to execute (e.g., automatically execute) the method 400.

First, the method 400 may include determining 402 contention for an instrument of a cell processing system (e.g., CPS 100). The instrument may be a subsystem and/or component of the CPS, such as at least a portion of one of the feedthrough 111, MHS 113, the RVS 118, the SLTS 115, SS 117, the BPS 119, the QCS 116, and the components 150 (e.g., one or more motors, sensors, heaters, lasers, pumps, and the like). In some variations, the determining 402 may be based on a number of cell samples having workflows that require use of the instrument (e.g., at least a portion of the instrument) for a same period of time (e.g., an overlapping range of seconds, minutes, or hours). Some instruments may be configured to simultaneously handle (e.g., perform an operation for or on) more cell samples than other instruments. As such, the contention for an instrument may be based on a unique number of cell samples in contention for the instrument, such as 2, 3, 4, 5, 6, 7, 8, 9, 10, or more than 10 cell samples. That is, high or excess contention may be determined when the number of cell samples requiring the instrument at the same time is over a predetermined threshold for the instrument (e.g., over 1, 2, 3, 4, 5, 6, 7, 8, 9, 10, or more than 10 cell samples). In some variations, the determining 402 may include determining contention for one or more instruments at once, such as for a plurality of instruments, including all instruments, of the system. In some variations, the determining 402 may occur before and/or after a cell processing operation of the system, such as before and after every cell processing operation executed by the system. Accordingly, the instrument contention may be recalculated continuously throughout a cell manufacturing process. In some variations, the method 400 may include generating a notification of excess contention in response to the determining 402.

When high or excess contention is determined, the method 400 may include evaluating 404 the priority restraints for the cell samples (e.g., all of the cell samples) in contention for the instrument. The priority restraints may be based on an operation dwell time (e.g., current operation dwell time) and/or an operation execution time (e.g., a subsequent operation execution time, which may be a minimum and/or maximum execution time) determined for each of the cell samples. As described herein (e.g., with respect to FIG. 2), the evaluating 404 may include comparing one or more of the operation dwell times and one or more of the operation execution times for the cell samples. This may determine a weight of instrument contention for each of the cell samples, where the weight of instrument contention may define how in-need of the instrument the cell sample is compared to all the others (based on the priority constraints of all of the cell samples). The weight of instrument contention may simply be a priority rank of the cell sample. For example, a cell sample with the largest weight contention may be ranked as the sample in the highest (most prioritized, number one) need for the instrument. Optionally, the method 400 may include reconfiguring/reschedule a workflow for one or more cell samples that were not identified as highest priority (e.g., will not use the instrument when planned).

Next, the method 400 may include identifying 406 one or more highest priority cell samples (prioritized samples for using the instrument) based on the evaluating 404. For example, the identifying 406 may include generating/providing an indication of the highest priority cell sample or the stack rank of cell samples based on weight contention for the instrument. The indication may be provided in graphical and/or numerical form via a display (e.g., display 126) and/or a user interface (e.g., user interface 128). In some variations, the identifying 406 may include indicating that the cell samples cannot be prioritized (e.g., all have equal or substantially equal weight contentions). In such variations, the method 400 may additionally include notifying or directing an operator to aid in resolving the contention, as described herein. In some variations, the operator may be able to manually reprioritize the cell samples for processing via the instrument based on priority information determined during the evaluating 404.

In cases where no high or excess contention is determined, the method may not include the evaluating 404, and may proceed to identifying 406 every cell sample within the system as a prioritized cell sample (e.g., may determine that every cell sample may proceed to be processed as scheduled/intended by its workflow).

In one example, a method for cell processing, including monitoring and/or managing contention within a cell processing system configured for parallel processing, may first include loading a plurality of cell samples into a cell processing system for parallel processing, where the cell processing system may include a plurality of processing instruments (e.g., subsystems). Next, the method may include determining an amount of contention for each of the plurality of instruments before and after each of a plurality of cell processing operations are executed by the cell processing system. The amount of contention may be based on a number of cell samples that need the instrument during a time period. Finally, the method may include generating a notification, via a user interface of the cell processing system, indicative of excess contention when the number of cell samples that need the instrument during the time period is greater than two. In some variations, the method may further include identifying a prioritized call sample to use the instrument based on a weight of instrument contention determined for each cell sample that needs the instrument during the time period, and the prioritized cell sample may have a largest weight of instrument contention. For each cell sample, the weight of instrument contention may be based on a current cell processing operation dwell time and a subsequent cell processing operation execution time.

The above-described methods can be implemented in any of numerous ways. For example, at least some methods of the present technology may be implemented using hardware, firmware, software, or a combination thereof. When implemented in firmware and/or software, the firmware and/or software code can be executed on any suitable processor or collection of logic components, whether provided in a single device or distributed among multiple devices.

In this respect, various aspects described herein may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods disclosed herein need not reside on a single computer or processor but may be distributed in a modular fashion amongst a number of different computers or processors to implement various aspects of the inventions disclosed herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in different variations.

Additionally, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Finally, the acts performed as part of the methods herein may be ordered in any suitable way. Accordingly, various methods may be constructed in which acts are performed in an order different than illustrated, which may include performing some acts simultaneously, even though shown as sequential acts in illustrative examples.

### EXAMPLES

### Example 1

FIG. 5 provides an exemplary simulated workflow for a cell sample. The workflow is depicted as a plot 500. Each cell processing operation is represented as a bar on the plot 500, where the length (execution time) of the operation correlates with a length of the corresponding bar. As shown, workflow starts December 25th at about 8 AM. A plurality of early cell processing operations were scheduled between about 8 AM to about 3 PM on December 25^{th}, (where a single operation is represented as a bar on the plot). Considering a load window of +/−1 hour, the cartridge may be loaded into the system any time between 7 AM to 9 AM. Without compensating for the load variation, this +/−1 hour window would impact the timing for all subsequent operations in the workflow. However, with the compensation, the load variation can be removed during the first extended processing step in the BPS. Here, the compensation was removed between about 4 PM and about 5 PM on December 26th. As a result, the cell processing steps beginning on December 27th at about 5 PM, including all remaining cell processing operations, were unaffected by variability in the operator load window timing through the end of the workflow.

Process variability compensation may be performed similarly to load variability compensation but also may occur throughout the entire workflow. For example, as shown on plot 500, the process variability that occurred on December 27^{th} between about 5 PM and about 7 PM was compensated for during the subsequent operation within the BPS, resulting in operations on December 28^{th} beginning between about 8 PM and about 9 PM.

### Example 2

FIGS. 6A-6B provide exemplary loading schedules 602, 604 for a cell processing system. The schedules 602, 604 show unavailable loading times for a proposed cartridge. The unavailable times are indicated by bars, each bar having a length that correlates to a length of the unavailable loading time period. Additionally, a shade of each bar—light, medium, or dark—indicates which subsystem or instrument of the workcell is at capacity, and thus preventing the proposed cartridge from loading during that time period. The dark bars represent the BPS at capacity, the medium bars represent the feedthrough being in-use or reserved for a scheduled cartridge, and the light bars represent contention within the SLTS that limits new inputs to the system.

The same simulated workflows were used for scheduled cartridges to generate the plots of FIGS. 6A and 6B, but the workflow for the proposed cartridge was varied between a 14-day (FIG. 3A) and 7-day (FIG. 3B) process. As it follows, the 7-day proposed cartridge workflow used to simulate the schedule 604 includes significantly fewer unavailable loading times, especially between January 10^{th} and January 25^{th}, during which time the manufacturing schedule of FIG. 3A was essentially completely unable to receive the proposed cartridge.

### Example 3

FIGS. 7A-7B provide additional exemplary loading schedules 702, 704 for a cell processing system. Like the schedules of 602, 604 of FIGS. 6A-6B, the schedules 702, 704 provide unavailable loading times indicated by bars, each bar having a length corresponding to a length of the unavailable loading time period. Additionally, a shade of each bar—light, medium, or dark—indicates which subsystem or instrument of the workcell is at capacity, and thus preventing the proposed cartridge from loading during that time period. The dark bars represent the BPS at capacity, the medium bars represent the feedthrough being in-use or reserved for a scheduled cartridge, and the light bars represent contention within the SLTS that limits new inputs to the system. As shown on schedule 702, a proposed cartridge having a 7-day workflow was added to the system at about 9:30 AM on December 31^{st}, an available loading time. Accordingly, the schedule 702 was updated to incorporate the newly scheduled cartridge, as shown in FIG. 7B. There, the updated schedule 704 shows the load window for the new cartridge (from about 9:30 AM to about 1 PM on the 31^{st}, as represented by the medium-shade bar). Additionally, the schedule 704 shows that, due to requirements of the workflow for the new cartridge, the BPS would be at capacity and/or would exceed capacity if another cartridge were loaded into the system between about 1 PM on December 31^{st} and about 12 AM on January 7^{th}. Additionally, the schedule of 704 changed the status of a significant amount of potential loading time from available to unavailable between December 25^{th} and December 31^{st} to prevent another cartridge from loading prior to the new cartridge of interest (which would cause the system to exceed capacity when the new cartridge of interest is loaded).

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

What is claimed is:

1. A method for cell processing comprising:
determining a system configuration of a cell processing system configured to process a plurality of cartridges in parallel, wherein the each of the plurality of cartridges is configured to carry a cell sample and to be loaded into a workcell of the cell processing system;
determining a cartridge configuration for each of the plurality of cartridges; and
providing a loading schedule for loading a first cartridge of the plurality of cartridges into the workcell, wherein the loading schedule is based on the system configuration and the cartridge configuration for each of the plurality of cartridges.

2. The method of claim 1, wherein the loading schedule indicates one or more time periods that are not available for loading the first cartridge into the workcell.

3. The method of claim 1 further comprising loading the first cartridge into the workcell based on the loading schedule.

4. The method of claim 1, wherein the loading schedule is provided via a user interface of the cell processing system.

5. The method of claim 1, wherein the system configuration defines a quantity of one or more subsystems of the cell processing system.

6. The method of claim 5, wherein the one or more subsystems are housed within the workcell of the cell processing system.

7. The method of claim 5, wherein the one or more subsystems comprise one or more of a materials handling system, a sterile liquid transfer system, a sterility system, a bioprocessing system, and a quality control system.

8. The method of claim 5, wherein the system configuration further defines an operational time constraint for at least one of the one or more subsystems of the cell processing system.

9. The method of claim 1, wherein the cartridge configuration comprises one or both of a loading configuration and a workflow configuration.

10. The method of claim 9, wherein the loading configuration comprises a length of time for loading a cartridge into the workcell.

11. The method of claim 9, wherein the workflow configuration comprises a simulated cell processing workflow for a cartridge, and wherein the simulated cell processing workflow is based on a process design plan for the cartridge and one or more sample intake parameters for the cartridge.

12. The method of claim 11, wherein the one or more sample intake parameters comprise one or more of a sample cell type, a sample collection time, a transport time for the sample, a processing facility arrival time for the sample, a sample temperature, patient age, patient sex, donor age, and donor sex.

13. The method of claim 1, wherein determining the cartridge configuration comprises padding data of the cartridge configuration to compensate for one or both of workflow process variability and cartridge loading variability.

14. The method of claim 1, wherein the plurality of cell processing cartridges comprises at least one second cartridge, and wherein the first cartridge is not scheduled for processing within the cell processing system and the at least one second cartridge is scheduled for processing within the cell processing system.

15. The method of claim 11 further comprising scheduling the first cartridge for processing within the cell processing system based on the loading schedule.

16. The method of claim 1 further comprising determining an amount of contention within the cell processing system, wherein the loading schedule is further based on the amount of contention determined.

17. A method for cell processing comprising:
loading a plurality of cell samples into a cell processing system for parallel processing, wherein the cell processing system comprises a plurality of processing instruments; and
determining an amount of contention for each of the plurality of instruments before and after each of a plurality of cell processing operations is executed by the cell processing system, wherein the amount of contention is based on a number of cell samples that need the instrument during a time period; and
generating a notification, via a user interface of the cell processing system, indicative of excess contention when the number of cell samples that need the instrument during the time period is greater than two.

18. The method of claim 17 further comprising identifying a prioritized cell sample of the plurality of cell samples to use the instrument based on a weight of instrument contention determined for each cell sample that needs the instrument during the time period.

19. The method of claim 18, wherein the prioritized cell sample comprises a largest weight of instrument contention.

20. The method of claim 18, wherein, for each cell sample, the weight of instrument contention is based on a current cell processing operation dwell time and a subsequent cell processing operation execution time.
